(19) United States
(12) Patent Application Publication
(10) Pub. No.: US 2010/0287635 A1
(43) Pub. Date: Nov. 11, 2010

(54) Cloned Canines and Method for Producing Thereof

(75) Inventors: Woo Suk Hwang, Seoul (KR); Byeong Chun Lee, Seoul (KR); Sung Keun Kang, Seoul (KR); Min Kyu Kim, Seoul (KR); Goo Jang, Seoul (KR); Hyun Ju Oh, Seoul (KR); Hye Jin Kim, Seoul (KR); Joung Ju Kim, Seoul (KR); Shamim Hossein Mohammad, Seoul (KR); Heru Fibrianto Yuda, Seoul (KR)

Correspondence Address: Juan Carlos A. Marquez;c/o Stites & Harbison PLLC, 1199 North Fairfax Street, Suite 900, Alexandria, VA, 22314-1437, US

(73) Assignee: SEOUL NATIONAL UNIVERSITY INDUSTRY FOUNDATION, Seoul (KR)

(21) Appl. No.: 11/989,502

(22) PCT Filed: Jul. 26, 2006
(86) PCT No.: PCT/KR2006/002938

§ 371 Date: Aug. 10, 2009

(30) Foreign Application Priority Data
Jul. 26, 2005 (KR) 10-2005-0067736

(51) Int. Cl. C12N 15/89; A01K 67/027; C12N 15/63

(52) U.S. Cl. 800/14; 800/24; 435/461

(57) ABSTRACT

Disclosed herein are a cloned canine and a production method thereof. The method comprises the steps of enucleating the oocyte of a canine to prepare an enucleated recipient oocyte, conducting nuclear transfer into the enucleated oocyte using a canine somatic cell as a nuclear donor cell under optimized conditions so as to prepare a nuclear transfer embryo, and transferring the nuclear transfer embryo into the oviduct of a surrogate mother. The present invention provides a method for producing cloned canines and thus, can contribute to the development of studies in veterinary medicine, anthropology and medical science such as the propagation of superior canines, the conservation of rare or nearly extinct canines, xenotransplantation and disease animal models.

### TECHNICAL FIELD

The present invention relates to a cloned canine and a production method thereof. More particularly, the present invention relates to a method for producing a cloned canine, comprising enucleating the mature oocyte of a canine to prepare an enucleated recipient oocyte, conducting nuclear transfer into the enucleated oocyte using a canine somatic cell as a nuclear donor cell under optimized conditions so as to prepare a nuclear transfer embryo, and transferring the nuclear transfer embryo into the oviduct of a surrogate mother, as well as a cloned canine produced by this method.

### BACKGROUND ART

With the recent development of somatic cell nuclear transfer technology by cell fusion or intracytoplasmic cell injection, the production of cloned animals is really conducted.

The somatic cell nuclear transfer technology, which is the technology allowing a living offspring to be born without undergoing meiosis and haploid germ cell formation which generally occur in a generative process, is a method of developing new individuals by transferring the diploid somatic cells of adults into enucleated cells to produce embryos and transferring the embryos in vivo. Generally, in the somatic cell nuclear transfer technology, recipient oocytes to be transferred with somatic cell donor nuclei are used after they are artificially cultured in vitro to metaphase II of meiosis. Then, in order to prevent the development of chromosomal abnormality resulting from somatic cell nuclear transfer, the mature oocytes are enucleated before transferring somatic cells. After injecting somatic cells into the perivitelline space or cytoplasm of the mature oocytes, the enucleated oocytes and the somatic cells are physically fused with each other by electrical stimulation. The fused couplet are activated by electrical stimulation or chemical substances and transferred into surrogate mothers to produce living offspring.

Such somatic cell nuclear transfer technology can be widely used in the field, for example in the propagation of superior animals, the conservation of rare or nearly extinct animals, the production of certain nutrients, the production of therapeutic bio-materials, the production of animals for organ transplantation, the production of animals with diseases or disorders the production of medically worthy animals for the substitution of organ transplantation such as a remedy of a cell and a gene.

Animal cloning technology was first accomplished by Dr. Wilmut of the Roslin Institute, England, by taking a mammary gland cell from a six-year old sheep, transferring the cell into an enucleated oocyte to prepare a nuclear transfer embryo, and transferring the embryo in vivo, thus producing a cloned animal, Dolly. Since then, cloned cows, mice, goats, pigs and rabbits have been produced by nuclear transplantation using somatic cells obtained from adult animals (WO 9937143 A2, EP 930009A1, WO 9934669A1, WO 9901164A1 and U.S. Pat. No. 5,945,577).

Meanwhile, not only the cloning of industrial animals, such as cows and pigs, but also the cloning of other pet animals such as dogs, attract the interest of many persons. Recently, among pet animals, a cat was first cloned, and a study on dog cloning was also conducted.

However, there is still no report showing that the cloning of canines by a somatic cell transfer method has succeeded.

### DISCLOSURE OF INVENTION

### Technical Problem

Accordingly, the present inventors have conducted studies on a production method of a cloned canine, and consequently, first produced cloned canines by a somatic cell transfer method under optimized conditions for electrical fusion, the activation of a nuclear transfer embryo and the transfer of embryo into a surrogate mother, thereby completing the present invention.

Therefore, it is an object of the present invention to provide a method for preparing a canine nuclear transfer embryo using somatic cell nuclear transfer technology.

Another object of the present invention is to provide a canine nuclear transfer embryo prepared by said method.

Still another object of the present invention is to provide a method for producing cloned canine, comprising the step of transferring said nuclear transfer embryo into surrogate mother to allow living offspring to be born.

Yet another object of the present invention is to provide a cloned canine produced by said method.

### Technical Solution

To achieve the above objects, in one aspect, the present invention provides a method for preparing a canine nuclear transfer embryo using somatic cell nuclear transfer technology.

In another aspect, the present invention provides a canine nuclear transfer embryo prepared by said method.

In still another aspect, the present invention provides a method for producing a cloned canine, comprising the step of transferring said nuclear transfer embryo into surrogate mother to allow living offspring to be born.

In yet another aspect, the present invention provides a cloned canine produced by said method.

Hereinafter, the present invention will be described in detail.

Term Definition

The term “nuclear transfer” as used herein refers to a gene manipulation technique, for having an identical characteristic form and quality acquired by artificially combining an enucleated cell with a nuclear DNA of one cell.

The term “nuclear transfer embryo” as used herein refers to an embryo injected or fused into/with a nuclear donor cell.

The term “cloned” as used herein refers to a gene manipulation technique preparing a new individual unit having an identical gene set with another individual unit. The term, particularly in present invention, is referred to the fact that a cell, an embryonic cell, a fetal cell, and/or an animal cell have a nuclear DNA sequence which is substantially similar or identical to a nuclear DNA sequence of another cell, the embryonic cell, the fetal cell, and/or the animal cell.

The term “nuclear donor cell” as used herein refers to a cell or a nucleus from a cell that is translocated into a recipient oocyte as a nuclear acceptor.

The term “recipient oocyte” as used herein refers to an oocyte that receiving the transfer of a nucleus from nuclear donor cell after its nucleus has been removed.

The term “mature oocyte” as used herein refers to an oocyte in metaphase II of meiosis.

The term “enucleated oocyte” as used herein refers to an oocyte which has had its nucleus removed.

The term “fusion” as used herein refers to combination between a nuclear donor and a lipid membrane of recipient oocyte. For example, the lipid membrane may be the plasma membrane or nuclear membrane of cells. The fusion can occur with addition of an electrical stimulation between a nuclear donor and recipient oocyte when they are placed adjacent to each other or when a nuclear donor is placed in the peri vitelline space of a recipient oocyte.

The term “activation” as used herein refers to stimulating a cell to divide, before, during or after the nuclear transfer step. Preferably, in the present invention, it means stimulating a cell to divide after the nuclear transfer step.

The term “living offspring” as used herein means an animal that survives ex utero. A “living offspring” animal may be an animal that is alive for at least one second, one minute, one day, one week, one month, six months or more than one year from the time it exits the maternal host. A “living offspring” animal may not require the circulatory system of an in utero environment for survival.

The term “canines” as used herein refers to include dogs, wolves, foxes, jackals, coyotes, Korean wolves and raccoon dogs. Preferably, they include dogs or wolves. The dogs are known to result from the domestication of wild wolves, and thus, they have the same chromosome number and show similarity in gestation period and sex hormone changes (Seal US et al., Biology Reproduction 1979, 21:1057-1066).

The present invention is characterized in that the cloning of a canine by somatic cell nuclear transfer technology was first successfully performed by preparing a canine nuclear transfer embryo under optimized conditions for the electrical fusion and activation of the nuclear transfer embryo and transferring the nuclear transfer embryo into the oviduct of a surrogate mother to produce a living offspring.

The inventive method for preparing a canine nuclear transfer embryo can comprise the steps of: (a) enucleating the mature oocyte of a canine to prepare an enucleated recipient oocyte; (b) isolating a somatic cell from the tissue of a donor canine to prepare a nuclear donor cell; (c) microinjecting the nuclear donor cell of the step (b) into the enucleated oocyte of the step (a) and electrically fusing the donor cell with the enucleated oocyte in a voltage of 3.0-3.5 kV/cm; and (d) activating the fused oocyte of the step (c).

Hereinafter, each step of the inventive method for producing canine nuclear transfer embryo will be described.

Step 1: Enucleation of Recipient Oocytes

For use as recipient oocytes, immature oocytes collected from canines can be matured in vitro, or oocytes matured in vivo can be collected. Generally, the oocytes of mammals (e.g., cattle, pigs and sheep) are ovulated in mature oocytes, i.e., metaphase II stage of meiosis, whereas canine oocytes are ovulated at prophase I stage of meiosis unlike other animals and matured while staying in the oviduct for 48-72 hours. Because the maturation rate of canine oocyte nucleus is very low and the ovulation time and reproductive physiology of canines are different from other animals, canine oocytes matured in vivo are preferably collected for use as recipient oocytes.

More specifically, the collection of mature oocytes from canines is preferably conducted at 48-72 hours and more preferably 72 hours after ovulation induction in the canines. In this regard, the day of ovulation in canines can be determined by any method known in the art. Examples of the method of determining the day of ovulation include, but are not limited to, vaginal smear tests, the measurement of serum sex hormones level, and the use of ultrasonographic diagnosis systems. The start of estrus in canines can be confirmed by vulva swelling and serosanguinous discharge.

In one example of the present invention, vaginal smear test and the analysis of serum progesterone concentration were conducted; the day on which nonkeratinized epithelial cells reached more than 80% and serum progesterone concentration reached about 4.0-7.5 ng/mL was regarded as the day of ovulation. On the basis of this determination, oocytes were collected at 48-72 hours and preferably 72 hours after ovulation. Meanwhile, maturation time of oocytes ovulated from canine is known to be 48-72 hours after ovulation; the present inventors analyzed oocytes collected at 48 hours, 60 hours and 72 hours after ovulation, and as a result, confirmed that oocytes collected at about 72 hours after ovulation are mature oocytes corresponding to metaphase II of meiosis. Also, an oocyte succeeding in actually producing a cloned dog in the present invention was an oocyte collected at 72 hours after ovulation. This suggests that it is most preferable to collect mature oocytes from canines at 72 hours after ovulation.

As a method of collecting oocytes matured in vivo, a surgical method including anesthetizing an animal followed by laparotomy can be used. More specifically, the collection of oocytes matured in vivo can be performed using salpingectomy by any method known in the art. The salpingectomy is a method of collecting the oocyte from the flushing by flushing downward an oocyte collection medium into the oviduct after surgically excising the oviduct.

In another method, oocytes matured in vivo can be collected by inserting a catheter into the fimbriated end of the oviduct, and injecting a flushing into the uterotubal junction using a needle indwelling catheter. This method has an advantage in that it does not cause damage the oviduct, and thus, allows an oocyte donor animal to be used for the next estrus.

Accordingly, the collection of oocytes matured in vivo is preferably preformed using the method including the use of the catheter that is not caused damage the oviduct. Meanwhile, in order to increase oocyte collection rate in the oocyte collection method including the use of the catheter, the present inventors have developed an oocyte retrieval needle which has a rounded front end such that it is easily inserted into the entrance of the oviduct (see FIG. 1). More specifically, a method of collecting oocytes using the needle developed by the present inventors comprises inserting and ligating the oocyte retrieval needle having a rounded front end in the oviduct, followed by flushing downward oocyte collection medium into the uterotubal junction so as to allow the flushing to flow into the oocyte retrieval needle, and observing the flushing with a microscope so as to select mature oocytes.

After the collection of mature oocytes, the haploid nuclei of the oocytes are removed. The enucleation of the oocytes can be performed by any method known in the art (see U.S. Pat. No. 4,994,384; U.S. Pat. No. 5,057,420; U.S. Pat. No. 5,945,577; EP Pat. No. 0930009 A1; Korean patent 342437; Kanda et al, J. Vet. Med. ScL, 57(4):641-646, 1995; Willadsen, Nature, 320:63-65, 1986, Nagashima et al., MoI. Reprod. Dev. 48:339-343 1997; Nagashima et al., J. Reprod Dev 38:37-78, 1992; Prather et al., Biol. Reprod 41:414-418, 1989, Prather et al., J. Exp. Zool. 255:355-358, 1990; Saito et al., Assis Reprod Tech Andro, 259:257-266, 1992; Terlouw et al., Theriogenology 37:309, 1992).

Preferably, the enucleation of recipient oocytes can be performed by either of the following two methods. One method comprises removing the cumulus cells of mature recipient oocytes, incising a portion of the zona pellucida of the recipient oocytes using a microneedle to give a slit, and removing the first polar body, nucleus and adjacent cytoplasm (the smallest possible amount) through the slit. Another method comprises removing the cumulus cells of recipient oocytes, staining the oocytes, and removing the first polar body and nucleus of the oocytes using an aspiration pipette. More preferably, for the enucleation of oocytes, the aspiration method is used for oocytes with a high survival rate, and the method of forming the slit is used for oocytes with low survival rate when the state of recipient oocytes is visually evaluated.

Step 2: Preparation of Nuclear Donor Cells

As nuclear donor cells, somatic cells derived from canines can be used. Specifically, somatic cells used in the present invention may be canine embryonic cells, fetal cells, juvenile cells, or adult cells, and preferably, originated from the tissue such as cumulus, skin, oral mucosa, blood, bone marrow, liver, lungs, kidneys, muscles and reproductive tract etc. that can be obtained from the adult cells. Examples of somatic cells which can be used in the present invention include, but are not limited to, cumulus cell, epithelial cell, fibroblast, neural cell, epidermal cell, keratinocyte, hematopoietic cell, melanocyte, chondrocyte, erythrocyte, macropharge, monocyte, muscle cell, B lymphocyte, T lymphocyte, embryonic stem cell, embryonic germ cell. More preferably, somatic cells which can be used in the present invention may include fetal fibroblast, adult fibroblast, and cumulus cell.

Furthermore, the nuclear donor cells used in the present invention may be those obtained by transforming wild-type somatic cells with certain genes by a gene transfer method or a gene targeting method. The gene transfer or gene targeting method can be easily practiced by any person skilled in the art because it is known in the art.

The somatic cells which are provided as the nuclear donor cells can be obtained by a method of preparing surgical samples or biopsy samples, and from the samples, single cells can be obtained by any method known in the art. For example, some of tissue from an animal to be cloned is aseptically incised to obtain a surgical sample or a biopsy sample, and the sample is minced, treated with trypsin and then cultured in tissue culture medium. After culturing for 3-4 days in the tissue culture medium, the growth of the cells on a culture dish is confirmed. When the cells completely grow, some of the tissue is frozen and stored in liquid nitrogen for later use, and the remnants are subcultured for use in nuclear transfer. The cells to be continuously cultured for use in nuclear transfer are subcultured up to 10 times so as to prevent the cells from growing excessively.

The tissue culture medium used as described above may be one known in the art, and its examples include TCM-199, and DMEM (Dulbecco's modified Eagle's medium).

Step 3: Microinjection and Fusion of Nuclear Donor Cells

The microinjection of nuclear donor cells into enucleated oocytes was performed by microinjecting the nuclear donor cells between the cytoplasm and zona pellucida of the enucleated oocytes by using a transfer pipette.

The enucleated oocytes microinjected with nuclear donor cells are electrically fused with nuclear donor cells, by using a cell Manipulator.

The electrical fusion can be performed with direct or alternating current. Preferably it can be performed in a voltage of 3.0-3.5 kV/cm, and more particularly, it can be performed 1-3 times in a direct current voltage of 3.0-3.5 kV/cm, for 10-30 D. Most preferably, it can be performed two times in a direct current voltage of 3.0-3.5 kV/cm for 20 D. If the voltage in the fusion is less than 3.0 kV/cm or more than 3.5 kV/cm, the fusion rate between the oocytes and the nuclear donor cells will be very low. The above-described voltage range in the electrical fusion is characterized in that it is higher than a voltage range in general electrical fusion known until now (1.7-2.0 kV/cm).

In one test example of the present invention, in order to determine the optimum voltage range in electrical fusion, nuclear transfer embryos microinjected with nuclear donor cells were electrically fused in different voltage ranges and examined for the fusion rate with a microscope (see Test Example X). As a result, it could be seen that the nuclear fusion rate was higher in high voltage than in low voltage, and the highest fusion rate of 75.2% was shown in a voltage range of 3.0-3.5 kV/cm (see Table 7).

The fusion of nuclear donor cells to oocytes by electrical stimulation can be performed in a fusion medium. The fusion medium used in the present invention may be a medium containing mannitol, MgSO, Hepes and BSA.

Step 4: Activation of Nuclear Transfer Embryos

Activation of the fused nuclear transfer embryos is a step of reactivating the temporarily paused cell-cycle. In order to reactivate the cell-cycle, the activation of cell signal delivery materials of pausing elements of cell-cycle such as MPF, MAP kitase etc. has to be reduced.

Generally, methods of activating the nuclear transfer embryos include an electrical method and a chemical method. In the present invention, it is preferable to activate the nuclear transfer embryos by the chemical method. The chemical method hastens activation of nuclear transfer embryos more than the electrical method. As the chemical method, there is a method of treating unclear transfer embryos with material such as ethanol, inositol trisphosphate (IP ), bivalency ion (e.g. Ca.⁺ or Sr⁺), microtubule inhibitors (e.g. cytochalasin B), bivalency ion ionophore and protein kinase inhibitors such as 6-dimethylaminopurine, protein synthesis inhibitors (e.g., cy-cloheximide), phorbol 12-myristate 13-acetate (PM A). Preferably, as the chemical method for the activation of nuclear transfer embryos, a method of treating the nuclear transfer embryos simultaneously or stepwise with calcium ionophore and 6-dimethylaminopurin can be used in the present invention. More preferably, the nuclear transfer embryos are treated with 5-10 μM calcium ionophore at 37-39° C. for 3-6 minutes and then with 1.5 mM-2.5 mM 6-dimethylaminopurin at 37-39° C. for 4-5 hours.

In one test example of the present invention, after the nuclear transfer embryos were activated by the electrical method and the chemical method, the nuclear transfer embryos were observed for their developmental stage, (see Test Example 3). As a result, it could be confirmed that the chemical activation enhance the developmental potential of the nuclear transfer embryos, and the activation of the nuclear transfer embryos by the chemical method allowed the nuclear transfer embryos to development to the morula stage (see Table 8).

Thus, the present invention provides canine nuclear transfer embryos prepared by the above-described method. By the present inventors, one of the canine nuclear transfer embryos prepared in one example of the present invention was named “Snuppy” (cloned canine embryo). And “Snuppy” (cloned canine embryo) has been deposited with an international depositary authority, KCTC (Korean Collection for Type Cultures; Korean Research Institute of Bioscience and Biotechnology, 52, Oun-dong, Yusong-gu, Daejeon, Korea) on Jul. 15, 2005, under the accession number KCTC 10831 BP.

The nuclear transfer embryos are freeze-stored and can be used after dissolution, if needed.

Furthermore, the canine nuclear transfer embryos according to the present invention can be used to produce cloned canines by transferring them into surrogate mothers to allow living offsprings to be born. Preferably, the transfer of the inventive nuclear transfer embryos into surrogate mothers is performed by transferring the oviduct of the surrogate mothers. The transfer can be performed by any method known in the art, and preferably, a catheter can be used to transfer the cloned embryos.

In one example of the present invention, cloned dogs, “Snuppy” and “NT-2#”, were first produced by transferring the inventive nuclear transfer embryos into the oviducts of surrogate mothers (see Example 6). However, one test example of the present invention showed that if the nuclear transfer embryos according to the present invention were transferred into the uterus of surrogate mothers, the surrogate mothers would not become pregnant (see Test Example 4). This suggests that the transfer of the nuclear transfer embryos in producing cloned dogs is preferably performed into the oviduct.

Meanwhile, in the transfer of the nuclear transfer embryos into surrogate mothers, the nuclear transfer embryos may be at the 1-cell, 2-cell or 4-cell stage. Also, the nuclear transfer embryos can be cultured in 25 D microdrops of mSOF overlaid with mineral oil until surrogate mothers are prepared.

Accordingly, the present invention provides cloned canines. The cloned canines have exactly the same genetic characteristics as nuclear donor cells or donors. In one example of the present invention, cloned dogs were produced according to the inventive method and analyzed for their genetic characteristics using microsatellite analysis (see Test Example 1). As a result, it could be seen that the cloned dogs according to the present invention had exactly the same genetic characteristics as nuclear donor cells or donors (see Table 6).

### Advantageous Effects A

s described hereinbefore, the present invention provides a method for producing cloned canines. Thus, the present invention can contribute to the development of studies in veterinary medicine, anthropology and medical science such as the propagation of superior canines, the conservation of rare or nearly extinct canines, xenotransplantation and disease animal models.

### BRIEF DESCRIPTION OF THE DRAWINGS

FIG. 1 is a photograph showing 15-gauge and 18-gauge needles for oocyte retrieval, which were used to collect oocytes from dogs in one example of the present invention.

FIG. 2 is a photograph showing a cloned dog, Snuppy, produced according to the inventive method and a donor dog (a), and cloned dog Snuppy and its surrogate mother (b).

### BEST MODE FOR CARRYING OUT THE INVENTION

Hereinafter, the present invention will be described in detail by examples. It is to be understood, however, that these examples are given for illustrative purpose only and are not construed to limit the scope of the present invention

### Example 1

### Collection of Recipient Oocytes from Dogs

Dogs used to retrieve recipient oocytes were 131 mixed breed female dogs aged 1-3 years, which were kept according to the standards established by the Seoul National University for Accreditation of Laboratory Animal Care. Ovulation timing was determined by performing a vaginal smear test and measuring serum progesterone concentration in estrus dogs. And mature oocytes were retrieved at 48-72 hours after ovulation.

In order to measure serum progesterone concentration, 3-5 ml of blood was collected everyday and centrifuged to obtain serum, and the serum was analyzed using a DSL-3900 ACTIVE Progesterone Coated-Tube Radioimmunoassay Kit (Diagnostic Systems Laboratories, Inc., TX). The day on which the progesterone concentration initially reached 4.0-7.5 ng/ml was considered as the day of ovulation. (Hase et al., J. Vet. Med. ScL, 62:243-248, 2000).

To perform the vaginal smear test, smears were obtained daily from the day the initial sign of proestrus. Smears were collected by inserting a swab into the lips of the vulva, then rolling them on a slide glass. After staining with a Diff-Quik staining (International chemical Co., Japan), the smears were examined with a microscope; the time at which superficial cells reached more than 80% of the epithelial cells cornified index (Evans J. M. et al., Vet. Rec, 7:598-599, 1970) was regarded as the time of ovulation.

The maturation time of ovulated oocytes is known to be 48-72 hours after ovulation. Thus, the present inventors retrieved oocytes at 48-72 hours after ovulation in the following manner.

First, female dogs which had reached the retrieval time of oocytes matured in vivo were administered with 0.05 mg/kg of atropin sulfate and 0.025 mg/kg of ace-promazine maleate and anesthetized by administering 5 mg/kg of ketamine. The anesthesia was maintained by administering isoflurane.

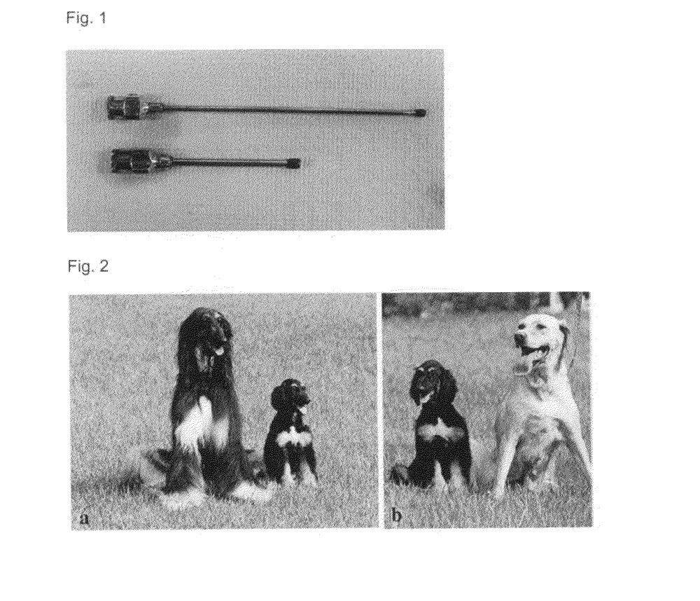

Anesthetized female dog was incised on an abdominal area by 5-10 D a And then the oocyte retrieval needle having a rounded front end (see FIG. 1) was inserted into the abdominal cavity of oviduct and held in place with suture, and then flushing downward oocyte collection medium (see Table 1) by attaching 24 gauge IV catheter into the uterotubal junction to flow the flushing into the 16 gauge needle. The flushing was transported into aseptic Petri-dish, and after that the flushing was observed with a microscope to select mature oocytes.

TABLE 1
Ooocyte collection medium

| Component | Content |
|---|---|
| TCM powder for 1 L (Gibco 31100-027) | 9.9 g |
| P/S antibiotics | 1% (10000 IU penicillin, |
| | 10 mg streptomycin) |
| HEPES buffer | 2.38 g |
| FBS | 10% (v/v) |
| NaHCO₃ | 0.1680 g |
| BSA | 5 mg/L |

As a result, an average 12 of mature oocytes per dog and a total 1370 of oocytes were collected.

### Example 2

### Enucleation of Recipient Oocytes

0.1% (v/v) hyaluronidase (Sigma, USA) was added to an hCR2aa medium (Table 2) prepared by adding Hepes-buffer to Ca⁺-free CR2 medium (Charles Rosenkrans 2; Rosenkrans et al., Biol. Reprod. 49, 459-462, 1993). Then, cumulus cells from the oocytes obtained in Example 1 were removed by repeated pipetting in the above medium. Then, the oocytes were stained with 5 D/mL bisbenzimide (Hoechst 33342) for 5 minutes and observed under an inverted microscope equipped with epiflu-orescence at 200× magnification so as to select only oocytes with the first polar body. 10% (v/v) FBS and 5 D/ml cytochalasin B were added to an hCR2aa medium (Table 2), and the selected oocytes were enucleated in the medium using a micromanipulator (Narishige, Tokyo, Japan). Namely, the oocytes were held with a holding micropipette (150 D inner diameter), and then the first polar body, adjacent cytoplasm (less than 5%) and oocyte nuclei were removed using an aspiration pipette. The enucleated oocytes were stored in a TCM-199 medium (Table 3) supplemented with 10% (v/v) FBS.

TABLE 2
Composition of hCR2aa medium

| Component | | | Content | |
|---|---|---|---|---|
| NaCl | | | 3.1 g/50 ml | |
| KCl | | | 0.1050 | g |
| KH₂PO₄ | | | 0.0230 | g |
| P/S | | | 5 | ml |
| Phenol-red | 400 [symbol] | mCR2-S | 4 | ml |
| NaHCO₃ | 1.0531 g/50 ml | St-B | 640 | [symbol] |
| HEPES | 0.5958 g/10 ml | St-E | 1680 | [symbol] |
| NEAA | | | 400 | [symbol] |
| Glycine | 0.0275 g/10 ml | Glycine | 400 | [symbol] |
| BSA | | | 0.12 | g |

TABLE 3
Composition of TCM-199 medium

| Component | Content | |
|---|---|---|
| TCM199 liquid | 89 | ml |
| pyruvic acid | 0.0099 | g |
| P/S (antibiotics) | 1 | ml |

| | |
|---|---|
| FBS | 10% |

### Example 3

### Preparation of Nuclear Donor Cells

As nuclear donor cells, adult fibroblasts collected from dogs were used. For this purpose, an ear skin biopsy from a three-year old male Afghan Hound was first isolated. Small pieces of the ear tissue fragment were washed three times in DPBS (Dulbecco's Phosphate Buffered Saline) and minced with a surgical blade. The minced tissue was dissociated in Dulbecco's modified Eagle's medium (DMEM; Life Technologies, Rockville, Md.) containing 0.25% (w/v) trypsin and 1 mM EDTA for 1 hour at 37° C. The trypsinized cells were washed once in Ca⁺- and Mg⁺-free DPBS by centrifugation at 300×g for 2 minutes, and seeded into 100-mm plastic culture dishes. The seeded cells were subsequently cultured for 6-8 days in DMEM supplemented with 10% (v/v) FBS, 1 mM glutamine, 25 mM NaHCO and 1% (v/v) minimal e ssential medium (MEM) nonessential amino acid solution (Life Technologies) at 39° C. in a humidified atmosphere of 5% CO and 95% air. After removal of unattached 2 clumps of cells or explants, attached cells were further cultured at intervals of 4 to 6 days by trypsinization for 1 min using 0.1% trypsin and 0.02% EDTA. Then, the subcultured cells were placed in a freezing medium and stored in liquid nitrogen at −196° C. The freezing medium consisted of 80% (v/v) DMEM, 10% (v/v) DMSO and 10% (v/v) FBS.

### Example 4

### Microinjection and Fusion of Nuclear Donor Cells into Enucleated Oocytes

The nuclear donor cells prepared in Example 3 were microinjected into the enucleated oocytes prepared in Example 2. After an aspiration pipette on the micromanipulator of Example 2 was replaced with a transfer pipette, the fixed oocytes were treated with 100 mg/mL of phytohemagglutinin in hCR2aa medium. The slit of the enucleated oocytes were held with a holding pipette and then inserted with a transfer pipette. Then, the single cells isolated from fibroblast in Example 3 were injected between the cytoplasm and zona pellucida of the enucleated oocytes by the transfer pipette.

The oocytes injected with the nuclear donor cells as described above were placed in a fusion medium (containing 0.26 M mannitol, 0.1 mM MgSO, 0.5 mM Hepes and 0.05% BSA), and transferred into a cell fusion chamber equipped with a stainless steel wire electrode (BTX 453, 3.2 mm gap; BTX, San Diego, Calif.). After equilibration for 3 minutes, the couplets were applied with direct current in a voltage of 3.0-3.5 kV/cM for 20 seconds using a BTX Electro-cell Manipulator, thus fusing the donor cells to the oocytes. The fusion was conducted in low voltage (close to 3.0 kV/cM) when the retrieved oocytes were weak oocytes. Also, when the oocytes were healthy oocytes, the fusion was conducted in high voltage (close to 3.5 kV/cM). The fusion was conducted at an average voltage of 3.3 kV/cm.

1,095 of fused nuclear transfer embryos were selected by a stereomicroscopic examination and cultured for 3 hours in modified synthetic oviductal fluid (mSOF) as shown in Table 4 (Jang et al., Reprod Fertil Dev, 15, 179-185, 2003).

TABLE 4
Composition of mSOF

| Component | | | Volume | |
|---|---|---|---|---|
| NaCl (54.44) | 2.900-3.100 g/ml | Stock-T | 2 | ml |
| KCl (74.55) | 0.2669 g | | | |
| KH₂PO₄ (136.1) | 0.0810 g | | | |
| Sod. Lactate | 0.28 ml | | | |
| Kanamycin | 0.0375 g | | | |
| Phenol-Red | 0.0050 g | | | |
| NaHCO₃ (84.01) | 1.0531 g/50 ml | Stock-B | 2 | ml |
| | 0.42124 g/20 ml | | | |
| Sod. Pyruvate (110.0) | 0.0182 g/5 ml | Stock-C | 200 | [symbol] |
| MgCl₂6H₂O (147.0) | 0.0996 g/10 ml | Stock-M | 200 | [symbol] |
| CaCl₂2H₂O (203.3) | 0.2514 g/10 ml | Stock-D | 200 | [symbol] |
| Glucose (180) | 0.27024 g/10 ml | | 200 | [symbol] |
| Glutamine (146.1) | 0.14618 g/10 ml | | 200 | [symbol] |
| Citri Acid (192) | 0.096 g/10 ml | Stock-CA | 200 | [symbol] |
| HEPES (238.3) | 0.5958 g/10 ml | Stock-E | 200 | [symbol] |
| EAA (Gibco 11051-018) | | | 400 | [symbol] |
| NEAA (Gibco | | | 200 | [symbol] |
| 11140-019) | | | | |
| ITS (I-3146) | | | 100 | [symbol] |
| BSA (fatty acid free) | | | 0.1600 | g |
| Hyaluronic Acid | | 0.5 mg/ml | | |
| 1N NaOH | | | | |
| D.W. | | | [symbol] 20 | ml |

pH 7.2-7.4
Osmolarity 275-285
EAA and NEAA requires care because of light sensitivity; and
The amount of phenol-red in the medium is insignificant because it is an indicator.

### Example 5

### Activation of Nuclear Transfer Embryos

The nuclear transfer embryos obtained in Example 4 were cultured in mSOF (Table 4) containing 1 OμM ionophore for 4 minutes at 39° C. The embryos were then washed and further incubated for 4 hours in mSOF supplemented with 1.9 mM of 6-dimethylaminopurine.

By the present inventors, one of the canine nuclear transfer embryos prepared as described above was named “Snuppy” (cloned canine embryo), and have been deposited with an international depositary authority, KCTC (Korean Collection for Type Cultures; Korean Research Institute of Bioscience and Biotechnology, 52, Oun-dong, Yusong-gu, Daejeon, Korea) on Jul. 15, 2005, under the accession number KCTC 10831 BP. The nuclear transfer embryos were cultured in 25 D microdrops of mSOF overlaid with mineral oil before embryos transfer into surrogate mothers.

### Example 6

### Embryo Transfer into Surrogate Mothers and Production of Cloned Dogs

The nuclear transfer embryos from Example 5 surgically transferred into the oviduct of surrogate mothers. The transfer was conducted depending on the preparation state of surrogate mothers after the activation of the nuclear transfer embryos in Example 5. Namely, when the surrogate mothers were immediately prepared, the transfer of the nuclear transfer embryos was immediately conducted, and if it was not so, the transfer was conducted on the day following the activation of the nuclear transfer embryos (reproduction embryo stage: 2 cell stage or 4 cell stage). As the surrogate mothers, 123 of dogs consisting of mixed breeding dogs and Labrador Retrievers were used. The selected dogs were disease-free, showed the repetition of the normal estrus cycle and had a normal uterine condition. 1,095 of reconstructed embryos from Example 5 were surgically transferred into the surrogate mothers. For this purpose, the surrogate mothers were anesthetized by vascular injection with 0.1 mg/kg acepromazine and 6 mg/kg propofol, and maintained at the anesthetized state using 2% isoflurane. Operation area of anesthetized female dog was aseptically operated and incised on center of abdomen by 5-10 D in a general laparotomy so as to expose the oviduct. The abdominal cavity was stimulated by hand to draw the ovarium, the oviduct and the uterus to the incision. The mesovarium of the drawn ovarium was carefully handled to recognize the opening of the oviduct, and a 3.5 F Tom cat catheter (Sherwood, St. Louis, Mo.) equipped with a 1.0 ml tuberculin syringe (Latex free, Becton Dickinson & CO. Franklin lakes, N.J. 07417) was inserted into the oviduct to secure a sufficient space in the front of the catheter. Then, the nuclear transfer embryos were injected into the oviduct through the catheter. Whether the nuclear transfer embryos were successfully injected was observed under a microscope, and 500 ml physiological saline containing an antibiotic was injected into the abdominal cavity. The abdominal suture was performed with an absorbable suture, and then, skin suture was performed. To prevent post-surgery infection, a broad range of antibiotic was injected for 3 days.

At 22 days after transferring the nuclear transfer embryos into the surrogate mothers, pregnancies were detected using a SONOACE 9900 (Medison Co. LTD, Seoul, Korea) ultrasound scanner with an attached 7.0 MHZ linear probe. Pregnancy was monitored by ultrasound every 2 weeks after initial confirmation. As a result, it was confirmed that three dogs had become pregnant. Among them, one was subsequently lost, and from one of the remaining two animals, the first cloned dog was delivered by caesarean section on 24 Apr. 2005, 60 days after the transfer of the nuclear transfer embryos. The birth weight was 530 g and the cloned puppy appears to be healthy. The cloned puppy was named “Snuppy” (Seoul National University puppy). From the remaining one animal, the second cloned dog delivered by caesarean section on 29 May 2005, 60 days after the transfer of the nuclear transfer embryos. The birth weight was 550 g and the cloned puppy appears to be healthy. The second cloned puppy was named “NT-2#”.

### MODE FOR THE INVENTION

### Test Example 1

### Examination of Genetic Identity of Cloned Dogs Produced according to the Present Invention

According to the present invention, the cloned puppy Snuppy and the NT-2# obtained in Example 6 were examined to check whether the cloned puppy Snuppy and the NT-2# were genetically identical to the donor dog Afghan Hound of nuclear donor cell in Example 3.

The genomic DNA of the cloned puppies, the donor dog, the surrogate recipients and nuclear donor fibroblasts was isolated. For this purpose, tissue fragments were obtained from the tail of the cloned puppies, and blood samples were collected from the donor dog and the surrogate mother. Each of the tissue fragments, the blood samples, and the fibroblasts were incubated with a lysis buffer [0.05 M Tris (pH 8.0), 0.05 M EDTA (pH 8.0), 0.5% SDS] supplemented with 400 D proteinase K overnight. Then, phenol extraction and ethanol precipitation were conducted to isolated genomic DNA from each sample.

The isolated genomic DNA samples were dissolved in 50 D TE and used to perform microsatellite analysis with eight canine specific markers [PEZOl, PEZ02, PEZ08, PEZ15 (see U.S. Pat. No. 5,874,217), REN162B09, REN105L03, REN165M10, FH2140 (see http://www.fhere.org/science/dog_genome/dog.html)] (Francisco, L. V. et al. Mamm. Genome 7, 359-362 1996; Neff, M. W. et al. Genetics. 151, 803-820, 1999; Richman, M. et al. J. Biochem. Biophys. Methods 47, 137-149, 2001; Denise, S. et al. Animal Genetics. 35, 14-17, 2004). The isolated genomic DNA as a template was PCR-amplified using fluorescently labeled locus-specific primers (Table 5) prepared based on the sequences of the known markers. The amplification products were analyzed with an automated DNA sequence analyzer (ABI 373: Applied Biosystems, Foster City, Calif.). The PCR reaction consisted of predenaturation at 94° C. for 1 min, followed of denaturation at 94° C. for 20 sec, annealing at 58° C. for 20 sec and extension at 74° C. for 20 sec by 30 cycles, and then post-extension at 74° C. for 5 min. Also, proprietary software (GeneScan and Genotyper; Applied Biosystems) was used to estimate the size of the PCR products.

TABLE 5
Primers used for PCR amplification

| | | |
|---|---|---|
| Primer | Sequence | SEQ ID NO: |

| | | | |
|---|---|---|---|
| PEZ01 | Sense | 5′-GGCTGTCACTTTTCCCTTTC-3′ | 1 |
| | Antisense | 5′-CACCACAATCTCTCTCATAAATAC-3′ | 2 |
| PEZ02 | Sense | 5′-TCCTCTCTAACTGCCTATGC-3′ | 3 |
| | Antisense | 5′- | 4 |
| | | GCCCTTGAATATGAACAATGACACTGT | |
| | | ATC-3′ | |
| PEZ08 | Sense | 5′-TATCGACTTTATCACTGTGG-3′ | 5 |
| | Antisense | 5′-ATGGAGCCTCATGTCTCATC-3′ | 6 |
| PEZ15 | Sense | 5′-CTGGGGCTTAACTCCAAGTTC-3′ | 7 |
| | Antisense | 5′-CAGTACAGAGTCTGCTTATC-3′ | 8 |
| REN162B09 | Sense | 5′-CAAACTTGACAGTCTTTTCAGGA-3′ | 9 |
| | Antisense | 5′-GCATTCAAGATGCACCAATG-3′ | 10 |
| REN105L03 | Sense | 5′-GGAATCAAAAGCTGGCTCTCT-3′ | 11 |
| | Antisense | 5′-GAGATTGCTGCCCTTTTTACC-3′ | 12 |
| REN165MI0 | Sense | 5′-AACAGCCAAATCATGGAAGC-3′ | 13 |
| | Antisense | 5′-AGCACCTCCATCCTTTCCTT-3′ | 14 |
| FH2140 | Sense | 5′-GGGGAAGCCATTTTTAAAGC-3′ | 15 |
| | Antisense | 5′-TGACCCTCTGGCATCTAGGA-3′ | 16 |

As a result, it could be found that the cloned dogs Snuppy and NT-2# produced according to the present invention were genetically completely identical to the donor dog Afghan Hound and the fibroblasts isolated from the donor dog. On the other hand, the cloned dogs of the present invention and the surrogate mothers (Labrador Retrievers or mixed breeding dogs) were genetically distinct from each other (Table 6).

TABLE 6
Analysis of canine-specific microsatellite loci

| | | | | | | | Surrogate | | | | | |
|---|---|---|---|---|---|---|---|---|---|---|---|---|
| | | | | | | | female | | | | Surrogate | |
| | Donor dog | | | | Cloned dog | | (Labrador | | Cloned dog | | female | |
| | (Afghan Hound) | | | | Snuppy | | Retriever; | | NT-2 | | (Mongrel; | |
| | Blood | | Nuclear donor | | (Tail tissue | | Blood | | (Tail tissue | | Blood | |
| Canine | leucocytes | | fibroblasts | | fragment) | | leucocytes) | | fragment) | | leucocytes) | |
| markers | Peak 1 | Peak 2 | Peak 1 | Peak 2 | Peak 1 | Peak 2 | Peak 1 | Peak 2 | Peak 1 | Peak 2 | Peak 1 | Peak 2 |
| PEZ01 | 110 | 118 | 110 | 118 | 110 | 118 | 118 | | 110 | 118 | 119 | 123 |
| PEZ02 | 123 | 230 | 123 | 230 | 123 | 230 | 114 | 126 | 123 | 230 | 126 | 134 |
| PEZ08 | 230 | | 230 | | 230 | | 232 | | 230 | | 215 | 219 |
| PEZ15 | 214 | | 214 | | 214 | | 208 | 217 | 214 | | 214 | 244 |
| REN162B09 | 191 | 195 | 191 | 195 | 191 | 195 | 181 | | 191 | 195 | 182 | 192 |
| REN105L03 | 235 | | 235 | | 235 | | 235 | 243 | 235 | | 248 | 252 |
| REN165M10 | 187 | 191 | 187 | 191 | 187 | 191 | 177 | 187 | 187 | 191 | 179 | 187 |
| FH2140 | 122 | | 122 | | 122 | | 131 | | 122 | | 121 | 131 |

### Test Example 2

### Optimization of Conditions for Electrical Fusion of Nuclear Donor Cells to Enucleated Oocytes

To optimize conditions for the electrical fusion of nuclear donor cells to enucleated oocytes, the nuclear donor cells were microinjected into the enucleated oocytes in the same manner as in Example 4, and the donor cells and the oocytes were fused to each other in varying voltage conditions of 1.7-1.9 kV/cm, 2.1-2.5 kV/cm, and 3.0-3.5 kV/cm. Then, the reconstructed embryos were examined for fusion with a stere-omicroscope.

As a result, it could be seen that the case of a voltage condition of 3.0-3.5 kV/cm showed that 203 nuclear transfer oocytes of 270 nuclear transfer oocytes (fusion rate of 75.2%) were fused. It is indicating that this condition is much higher in fusion efficiency than other conditions (Table 7).

TABLE 7
Fusion rate of nuclear transfer oocytes according
to voltage condition in electrical fusion

| | | Number of fused |
|---|---|---|
| Number of oocytes used | Voltage condition | oocytes |
| 30 | 1.7~1.9 kV/cm | 10 (33.3%) |
| 50 | 2.1~2.5 kV/cm | 22 (44.0%) |
| 270 | 3.0~3.5 kV/cm | 203 (75.2%) |

### Test Example 3

### Optimization of Conditions for Activation of Nuclear Transfer Embryos

The nuclear transfer embryos obtained in Example 4 were activated by the electrical method and the chemical method. And then the nuclear transfer embryos were observed for their development stage. In the electrical method, the nuclear transfer embryos of Example 4 were placed in mannitol medium (containing 0.26 M mannitol, 0.1 mM MgSO, 0.5 mM Hepes and 0.05% BSA) with CaCl 10 OnM and transferred into a cell fusion chamber equipped with a stainless steel wire electrode (BTX 453, 3.2 mm gap; BTX, San Diego, Calif.). After equilibration for 3 minutes, the couplets were applied with direct current in a voltage of 3.0-3.5 kV/cM for 20 seconds using a BTX Electro-cell Manipulator, thus fusing the donor cells to the oocytes.

In the chemical activation method, the nuclear transfer embryos of Example 4 were placed in mSOF containing 10 mM ionophore (Sigma) and cultured in the medium at 39° C. for 4 minutes. Then, the culture was washed and further cultured in mSOF (Table 4) supplemented with 1.9 mM 6-dimethylaminopurin for 4 hours. After completion of the culture, the embryos were transferred into TCM 199 medium (Table 3).

Each developmental stage of the nuclear transfer embryos activated by the electrical method and the chemical method was examined using a stereomicroscope at 100× magnification.

As a result, it could be confirmed that the chemical activation enhance the developmental potential of the nuclear transfer embryos. Namely, it was shown that, in the case of the nuclear transfer embryos activated by the chemical method, 80% of the oocytes reached the 2-cell stage, but in the case of the nuclear transfer embryos the electrical method, only about 53% of the oocytes reached the 2-cell stage. Also, it could be found that the chemically activated embryo showed the development of the nuclear transfer embryos to the morula stage, but the electrically activated embryo showed development only to the 16-cell stage (Table 8).

TABLE 8
Developmental stage of nuclear transfer embryos according to the
method of activation

| | | Divi- | | | | | |
|---|---|---|---|---|---|---|---|
| | Number | sion | | | 16- | | |
| | of | (2-cell | 4-cell | 8-cell | cell | Morula | Blastocyte |
| Activation | oocytes | stage) | stage | stage | stage | stage | stage |
| Chemical | 50 | 40 | 20 | 16 | 8 | 2 | |
| activation | | | | | | | |
| Electrical | 40 | 21 | 8 | 5 | 1 | | |
| activation | | | | | | | |

### Test Example 4

### Optimization of Conditions for Transfer of Inventive Canine Nuclear Transfer Embryos into Surrogate Mothers

The nuclear transfer embryos activated in Example 5 were cultured in mSOF (Table 4) in an incubator at 38-39° C. and an atmosphere of 5% CO and 5% oxygen. Then, the embryos grown to the 8-cell stage were immersed in PBS containing 0.1% bovine fetal serum and transferred into the uterine cornual of 20 surrogate mothers (mixed breeding dogs) by a straw.

At 22 days after transferring the nuclear transfer embryos, pregnancies were detected using an ultrasound scanner (Medison Co. LTD, Seoul, Korea) according to the same manner as in Example 6.

As a result, it was found that none of the nuclear transfer embryos transferred into the uterus led to pregnancy. This suggests that it is preferable to transfer the nuclear transfer embryos into the oviduct as described in Example 6.

### INDUSTRIAL APPLICABILITY

As described hereinbefore, the present invention provides a method for producing cloned canines. Thus, the present invention can contribute to the development of studies in veterinary medicine, anthropology and medical science such as the propagation of superior canines, the conservation of rare or nearly extinct canines, xenotransplantation and disease animal models.

1. A method for preparing a canine nuclear transfer embryo, comprising the steps of:
(a) enucleating the mature oocyte of a canine to prepare an enucleated recipient oocyte;
(b) isolating a somatic cell from the tissue of a donor canine to prepare a nuclear donor cell;
(c) microinjecting the nuclear donor cell of the step (b) into the enucleated oocyte of the step (a) and electrically fusing the donor cell with the enucleated oocyte in a voltage of 3.0-3.5 kV/cm; and
(d) activating the fused oocyte of the step (c).

2. The method according to claim 1, wherein the mature oocyte is in vivo matured oocyte.

3. The method according to claim 2, the in vivo matured oocyte is retrieved from the canine at 48-72 hours after ovulation.

4. The method according to claim 1, wherein the somatic cell in the step (a) is one selected from the group consisting of cumulus cell, epithelial cell, fibroblast, neural cell, epidermal cell, keratinocyte, hematopoietic cell, melanocyte, chondrocyte, erythrocyte, macropharge, monocyte, muscle cell, B-lymphocyte, T-lymphocyte, embryonic stem cell and embryonic germ cell, fetal cell, placental cell and embryo cell.

5. The method according to claim 1, wherein the somatic cell is a fibroblast or a cumulus cell.

6. The method according to claim 1, wherein the electrically fusing in the step (c) is conducted 1-3 times in a direct current voltage of 3.0-3.5 kV/cm for 10-30 D.

7. The method according to claim 1, wherein the activating step in the step (d) is performed by treating the fused oocyte simultaneously or stepwise with calcium ionophore and DMAP (6-dimethylaminopurine).

8. The method according to claim 7, wherein the activation method is performed by treating the fused oocyte with 5-10 μM calcium ionophore at 37-39° C. for 3-5 minutes and then with 1.5 mM-2.5 mM DMAP (6-dimethylaminopurine) at 37-39° C. for 4-5 hours.

9. The method according to claim 1, wherein the canine is selected from the group consisting of dog, wolf, fox, jackal, coyote, Korean wolf and raccoon dog.

10. The method according to claim 1, wherein the canine is selected from the group consisting of dog, wolf and fox.

11. A nuclear transfer embryo prepared by a method as set forth in claim 1.

12. The nuclear transfer embryo of claim 11, which is deposited at KCTC (Korean Collection for Type Cultures) under accession number KCTC 1083 IBP.

13. A method for producing a canine, comprising the step of transferring the nuclear transfer embryo of claim 11 into the oviduct of a surrogate mother to allow a living offspring to be born.

14. The method according to claim 13, wherein the canine is selected from the group consisting of dog, wolf, fox, jackal, coyote, Korean wolf and raccoon dog.

15. The method according to claim 13, wherein the canine is selected from the group consisting of dog, fox and wolf.

16. A cloned canine produced by a method as set forth in claim 13.

17. The cloned canine according to claim 16, wherein the cloned canine has the same genotype as the nuclear donor cell or donor animal of claim 1.

18. The cloned canine according 16, wherein the canine is selected from the group consisting of dog, wolf, fox, jackal, coyote, Korean wolf and raccoon dog.

19. A method for producing a canine, comprising the step of transferring the nuclear transfer embryo of claim 12 into the oviduct of a surrogate mother to allow a living offspring to be born.


SEQUENCE LISTING (16 sequences)

SEQ ID NO: 1 - DNA, 20 bp, Artificial Sequence
  PEZ01 sense primer
ggctgtcacttttccctttc

SEQ ID NO: 2 - DNA, 24 bp, Artificial Sequence
  PEZ01 antisense primer
caccacaatctctctcataaatac

SEQ ID NO: 3 - DNA, 20 bp, Artificial Sequence
  PEZ02 sense primer
tcctctctaactgcctatgc

SEQ ID NO: 4 - DNA, 30 bp, Artificial Sequence
  PEZ02 antisense primer
gcccttgaatatgaacaatgacactgtatc

SEQ ID NO: 5 - DNA, 20 bp, Artificial Sequence
  PEZ08 sense primer
tatcgactttatcactgtgg

SEQ ID NO: 6 - DNA, 20 bp, Artificial Sequence
  PEZ08 antisense primer
atggagcctcatgtctcatc

SEQ ID NO: 7 - DNA, 21 bp, Artificial Sequence
  PEZ15 sense primer
ctggggcttaactccaagttc

SEQ ID NO: 8 - DNA, 20 bp, Artificial Sequence
  PEZ15 antisense primer
cagtacagagtctgcttatc

SEQ ID NO: 9 - DNA, 23 bp, Artificial Sequence
  REN162B09 sense primer
caaacttgacagtcttttcagga

SEQ ID NO: 10 - DNA, 20 bp, Artificial Sequence
  REN162B09 antisense primer
gcattcaagatgcaccaatg

SEQ ID NO: 11 - DNA, 21 bp, Artificial Sequence
  REN105L03 sense primer
ggaatcaaaagctggctctct

SEQ ID NO: 12 - DNA, 21 bp, Artificial Sequence
  REN105L03 antisense primer
gagattgctgccctttttacc

SEQ ID NO: 13 - DNA, 20 bp, Artificial Sequence
  REN165M10 sense primer
aacagccaaatcatggaagc

SEQ ID NO: 14 - DNA, 20 bp, Artificial Sequence
  REN165M10 antisense primer
agcacctccatcctttcctt

SEQ ID NO: 15 - DNA, 20 bp, Artificial Sequence
  FH2140 sense primer
ggggaagccatttttaaagc

SEQ ID NO: 16 - DNA, 20 bp, Artificial Sequence
  FH2140 antisense primer
tgaccctctggcatctagga